(19) United States
(12) Patent Application Publication
(10) Pub. No.: US 2021/0282737 A1
(43) Pub. Date: Sep. 16, 2021

(54) MEDICAL SILICA GEL STETHOSCOPE AND THE MANUFACTURING METHOD AND PROCESSING MOLD THEREOF

(71) Applicants: Fengsen Zhu, Wenzhou City (CN); Mingrong Wang, Wenzhou City (CN)

(72) Inventors: Fengsen Zhu, Wenzhou City (CN); Mingrong Wang, Wenzhou City (CN)

(21) Appl. No.: 17/198,290

(22) Filed: Mar. 11, 2021

(30) Foreign Application Priority Data
Mar. 11, 2020 (CN) 202010167769 .8

(51) Int. Cl. A61B 7/02; B29C 45/00; B29C 45/26

(52) U.S. Cl. CPC A61B 7/02; B29C 45/0001; B29K 2083/00; B29C 45/0053; A61B 2562/12; B29C 45/261

(57) ABSTRACT

A medical silica gel stethoscope, including a sound conducting pipe, the sound conducting pipe includes a U-section and a straight pipe section, said U-section and straight pipe section are molded integrally, and its manufacturing method thereof. The present invention also relates to a processing mold for said medical silica gel stethoscope. Technical effects of the present invention: 1) the U-section and straight pipe section of medical silica gel stethoscope of the present invention are molded integrally, so the inner wall of the pipe is free of seams, the sound conduction effect is better. The molding of the traditional splicing molded medical silica gel stethoscope requires at least three molds, whereas the one-step molded structure of the medical silica gel stethoscope of the present invention is easier for processing.

### BACKGROUND OF INVENTION

### 1. Field of the Invention

The present invention relates generally to a medical silica gel stethoscope and the manufacturing method and processing mold thereof.

### 2. Description of Related Art

The sound conducting pipe of traditional medical stethoscope includes a sound conducting section of three-way pipe and a straight pipe section. The interface end of the sound conducting section of three-way pipe and the interface end of the straight pipe section are set as one piece, and a seam is formed at the interface, the seam is a weld seam. The sound conducting section of three-way pipe and the straight pipe section are made of silica gel, rubber, emulsion or TPU material. However, the sound conducting pipe of traditional medical stethoscope has worse sound conduction effect, and the processing thereof is complicated and inefficient.

### SUMMARY OF THE INVENTION

The technical problem to be solved by the present invention is to provide a medical silica gel stethoscope with better sound guiding effect and more convenient processing. The technical problem to be solved by the present invention also includes providing a method for manufacturing the above-mentioned medical silica gel stethoscope. The technical problem to be solved by the present invention also includes providing a processing mold for the above-mentioned medical silica gel stethoscope.

For this reason, the medical silica gel stethoscope provided by the present invention includes a sound conducting pipe, the sound conducting pipe includes a U-section and a straight pipe section, its features are: the U-section and straight pipe section are molded integrally.

More particularly, a corrugate pipe is arranged in the straight pipe section.

More particularly, a three-way pipe is embedded in the joint of the U-section and straight pipe section.

More particularly, an inner pipe is embedded in the U-section, the inner pipe is filled with a filler to support the pattern of inner pipe, the filler is withdrawn from the inner pipe by dissolution, pumping or ejection after the U-section is molded by pouring, both ends of the inner pipe are connected to the three-way pipe and earhook respectively, the inner tube is a corrugate pipe, so that the silica gel of U-section is filled in the gaps and inner wall of corrugate pipe.

More particularly, a straight pipe corrugate pipe is embedded in the inner wall of the straight pipe section, so that the silica gel of straight pipe section is filled in the gaps and inner wall of straight pipe corrugate pipe, a left corrugate pipe and a right corrugate pipe are embedded in the right and left sides of the U-section respectively, so that the silica gel of U-section is filled in the gaps and inner walls of left corrugate pipe and right corrugate pipe, a three-way pipe is embedded in the joint of the U-section and straight pipe section, the inner end of the left corrugate pipe contacts the left end of three-way pipe, and the outer end contacts the inner end of earhook, the inner end of the right corrugate pipe contacts the right end right end of three-way pipe, and the outer end contacts the inner end of earhook, the earhook includes an earhook coupling end and an earhook body, the earhook coupling end is embedded in the U-section, the earhook body is screwed with the earhook coupling end, an annular step is arranged in the inner bore of earhook coupling end, a rubber sealing ring is arranged on the annular step, the rubber sealing ring is clamped between the earhook body and earhook coupling end, the earhook pipe of the earhook body is provided with a nut to adjust the earhook locking position and direction. The nut is located in the upside of earhook coupling end.

A manufacturing method for the medical silica gel stethoscope includes the following steps:

firstly, installation of sound conducting pipe mold: the straight pipe section core-pulling rod is inserted into the straight pipe section of sound conducting pipe mold, the outer end of straight pipe section core-pulling rod is fixed to the outside of sound conducting pipe mold by a lower fixing device, a U-core-pulling mold is inserted into the U-section of sound conducting pipe mold, both ends of U-core-pulling mold are fixed to the upper fixing device outside the mold, the end of the straight pipe section core-pulling rod is inserted into the plug hole of U-core-pulling mold, the sound conducting pipe mold is clamped, a pouring space for silica gel pipe remains between the U-core-pulling mold and the inner wall of sound conducting pipe mold;

secondly, molding of sound conducting pipe: the softened or liquid silica gel is injected to fill up the mold cavity with silica gel;

thirdly, the sound conducting pipe mold is removed after cooling, the straight pipe section core-pulling rod and U-core-pulling mold are pulled out, the surface of the molded sound conducting pipe is polished;

fourthly, the earhooks are inserted into both ends of U-section, the stethoscope is connected to the end of straight pipe section.

More particularly, a threaded hole is arranged in the downside in the middle of U-core-pulling mold in Step 1, the end of the straight pipe section core-pulling rod has a threaded connection end with external thread matching the threaded hole, the threaded connection end of the straight pipe section core-pulling rod enters the sound conducting pipe mold and enters the threaded hole to form threaded connection with the U-core-pulling mold.

More particularly, the U-core-pulling mold includes a left core-pulling mold and a right core-pulling mold, the ends of left core-pulling mold and right core-pulling mold form inserting connection, the inserting connection ends of the left core-pulling mold and right core-pulling mold are provided with a set of magnetic attraction mechanisms which can attract each other.

More particularly, a three-way pipe is embedded in the joint of the straight pipe section and U-section, two inner pipes are arranged in the U-section, both ends of inner pipe are connected to the three-way pipe and earhook respectively, during processing, the first step is to directly place the left and right inner pipes and three-way pipe in the sound conducting pipe mold, the inner pipe is filled with a filler to maintain the shape of inner pipe, and the silica gel is filled in between the inner pipe and three-way pipe and mold, cooled and molded, the filler is removed from the inner pipe after the sound conducting pipe mold is removed.

A processing mold for the medical silica gel stethoscope includes a sound conducting pipe mold, the sound conducting pipe mold includes an upper mold, a lower mold, a straight pipe section core-pulling rod and a U-core-pulling mold, the end of straight pipe section of the sound conducting pipe mold is provided with a lower plug, both ends of the U-section are provided with an upper plug respectively, the lower end of the straight pipe section core-pulling rod is provided with a fixing device, both ends of the U-core-pulling mold are provided with a fixing device respectively, the lower fixing device is a lower plug, the straight pipe section core-pulling rod is arranged on the lower plug, the upper end of the straight pipe section core-pulling rod is provided with a threaded connection end, the upper fixing device is an upper plug, both ends of the U-core-pulling mold are arranged on the upper plug respectively, a threaded hole is arranged in the downside in the middle of the U-core-pulling mold, the end of the straight pipe section core-pulling rod has a threaded connection end with external thread matching the threaded hole, the threaded connection end of the straight pipe section core-pulling rod enters the sound conducting pipe mold and enters the threaded hole to form threaded connection with the U-core-pulling mold, the lower end of the straight pipe section core-pulling rod is fixed to the lower plug, both ends of the U-core-pulling mold are fixed to the lower plug, the U-core-pulling mold includes a left core-pulling mold and a right core-pulling mold, the ends of left core-pulling mold and right core-pulling mold form inserting connection, the inserting connection ends of the left core-pulling mold and right core-pulling mold are provided with a set of magnetic attraction mechanisms which can attract each other.

The technical problem to be solved by the present invention is to provide a medical silica gel stethoscope more convenient for processing with better sound conduction effect, and to provide the manufacturing method and processing mold thereof.

### Technical Effects of the Present Invention

1. The U-section and straight pipe section of the medical silica gel stethoscope of the present invention are molded in one, so there is no seam on the inner wall of pipe, the sound conduction effect is better. The molding of the traditional splicing molded medical silica gel stethoscope requires at least three molds, whereas the one-step molded structure of the medical silica gel stethoscope of the present invention is easier to be processed.

2. The core-pulling mold of the present invention includes a straight pipe section core-pulling rod and a left core-pulling mold and a right core-pulling mold that form a U-core-pulling mold, convenient for performing pouring and core-pulling processes for U-section, preventing excessive curvature from failing core pulling.

3. The lower end of the straight pipe section core-pulling rod in the present invention is fixed to the lower plug. Both ends of the U-core-pulling mold are fixed to the lower plug, the core-pulling rod is fixed by the plug, the structure is simplified, and the processing operation is more convenient.

4. The threaded connection end of the straight pipe section core-pulling rod enters the sound conducting pipe mold and enters the threaded hole to form threaded connection with the U-core-pulling mold, so that the internal core-pulling mold forms a fixed integer, favorable for the stability of setting internal core-pulling mold.

5. The left core-pulling mold and right core-pulling mold ends form an inserting connection, the insertion connected ends of the left core-pulling mold and right core-pulling mold are provided with a set of magnetic attraction mechanisms which attract each other, so that the left core-pulling mold and right core-pulling mold are connected stably and firmly, and easy to be split, favorable for increasing the processing efficiency.

6. The inner pipe is filled with a filler to support the pattern of inner pipe. The filler is withdrawn from the inner pipe by dissolution, pumping or ejection after the U-section is molded by pouring. The structure supports the inner rod with filler, avoids using U-core-pulling rod, and avoids difficult U-pipe core-pulling.

### BRIEF DESCRIPTION OF THE DRAWINGS

FIG. 1 is a structural section view of the sound conducting pipe of medical silica gel stethoscope in the Embodiment 1 of the present invention;

FIG. 2 is a structural section view of the sound conducting pipe of medical silica gel stethoscope in the Embodiment 2 of the present invention;

FIG. 3 is a structural representation of processing mold for the sound conducting pipe of medical silica gel stethoscope in the Embodiment 1 of the present invention;

FIG. 4 is a structural representation of processing mold for the sound conducting pipe of medical silica gel stethoscope in the Embodiment 2 of the present invention;

FIG. 5 is the split structure diagram of U-core-pulling mold in FIG. 4.

FIG. 6 is a structural representation of processing mold for the sound conducting pipe of medical silica gel stethoscope in the Embodiment 3 of the present invention;

FIG. 7 is a structural section view of the sound conducting pipe of medical silica gel stethoscope in the Embodiment 4 of the present invention;

FIG. 8 is a structural section view of the sound conducting pipe of medical silica gel stethoscope in the Embodiment 5 of the present invention.

FIG. 9 is a close-up view of FIG. 8;

FIG. 10 is a structural section view of the high quality medical silica gel stethoscope in the Embodiment 6 of the present invention;

FIG. 11 is a close-up view of FIG. 10.

### DETAILED DESCRIPTION OF THE INVENTION

First of all, the words “front”, “back”, “left”, “right”, “up” and “down” used in the description refer to the directions in the attached figures, the words “bottom surface” and “top surface”, “inside” and “outside” refer to the directions toward or far from the geometric center of specific components respectively.

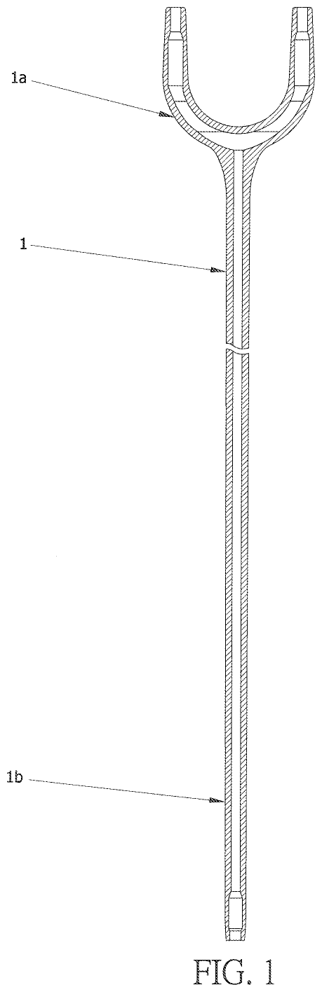

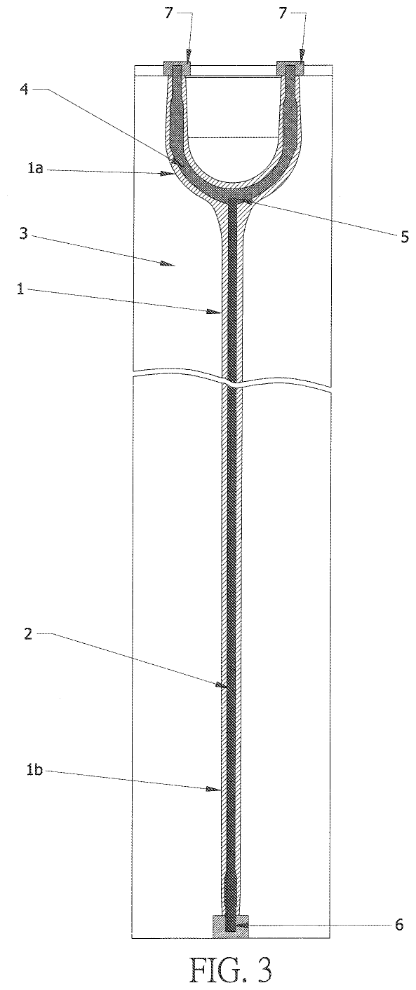

As shown in FIG. 1 and FIG. 3, the medical silica gel stethoscope provided in the Embodiment 1 of the present invention includes a sound conducting pipe 1, the sound conducting pipe 1 is made of silica gel, the sound conducting pipe 1 includes a U-section 1a and a straight pipe section 1b, the U-section 1a and straight pipe section 1b are molded integrally.

According to Embodiment 1, a method for manufacturing the medical silica gel stethoscope is provided, including the following steps:

Firstly, installation of sound conducting pipe mold: the straight pipe section core-pulling rod 2 is inserted into the straight pipe section 3a of sound conducting pipe mold 3, the outer end of straight pipe section core-pulling rod 2 is fixed to the outside of sound conducting pipe mold 3 with a lower fixing device. A U-core-pulling mold 4 is inserted into the U-section 3b of sound conducting pipe mold 3, both ends of U-core-pulling mold 4 are fixed to the upper fixing device outside the mold. The end of the straight pipe section core-pulling rod 2 is inserted into the plug hole 5 of U-core-pulling mold 4. The sound conducting pipe mold 3 is clamped, a pouring space for silica gel pipe remains between the U-core-pulling mold 4 and the inner wall of sound conducting pipe mold 3.

Secondly, molding of sound conducting pipe 1: the softened or liquid silica gel is injected to fill up the mold cavity with silica gel.

Thirdly, the sound conducting pipe mold 3 is dismounted after cooling, the straight pipe section core-pulling rod 2 and U-core-pulling mold 3 are pulled out, the surface of the molded sound conducting pipe 1 is polished.

Fourthly, the earhooks are inserted into both ends of U-section, the stethoscope is connected to the end of straight pipe section.

As shown in FIG. 3, the present invention provides a processing mold for the medical silica gel stethoscope, including a sound conducting pipe mold 3, the sound conducting pipe mold 3 sound conducting pipe mold 3 includes an upper mold, a lower mold, a straight pipe section core-pulling rod 2 and a U-core-pulling mold 4. The end of straight pipe section 3a of the sound conducting pipe mold 3 is provided with a lower plug 6, both ends of the U-section 3b are provided with an upper plug 7 respectively. The lower end of the straight pipe section core-pulling rod 2 is provided with a fixing device. Both ends of the U-core-pulling mold 4 is provided with a fixing device respectively. The lower fixing device is the lower plug 6. The straight pipe section core-pulling rod 2 is arranged on the lower plug 6. The upper end of the straight pipe section core-pulling rod 2 is provided with a threaded connection end. The upper fixing device is the upper plug 7. Both ends of the U-core-pulling mold 4 are arranged on the upper plug 6 respectively. The lower side in the middle of the U-core-pulling mold 4 is provided with a threaded hole 8. The end of the straight pipe section core-pulling rod 2 has a threaded connection end with external thread matching the threaded hole 8. The threaded connection end of the straight pipe section core-pulling rod 2 enters the sound conducting pipe mold and enters the threaded hole 8 to form threaded connection with the U-core-pulling mold 4. The lower end of the straight pipe section core-pulling rod 2 is fixed to the lower plug 6. Both ends of the U-core-pulling mold 4 are fixed to the upper plug 7.

The U-core-pulling mold 4 includes a left core-pulling mold 4a and a right core-pulling mold 4b. The ends of left core-pulling mold 4a and right core-pulling mold 4b form inserting connection. The inserting connection ends of the left core-pulling mold 4a and right core-pulling mold 4b are provided with a set of magnetic attraction mechanisms 9 which can attract each other.

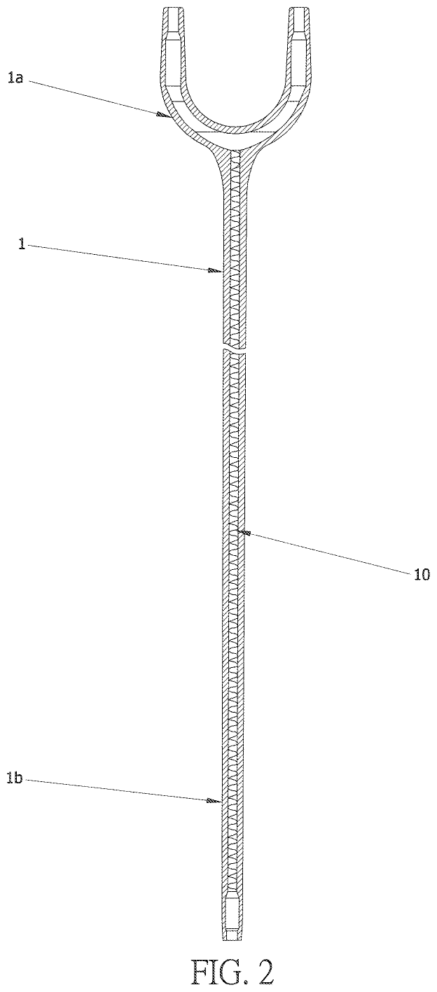

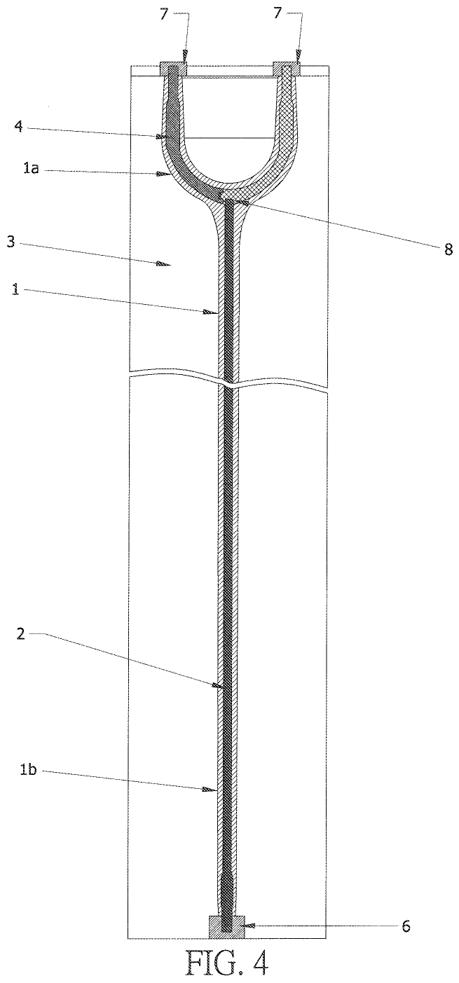

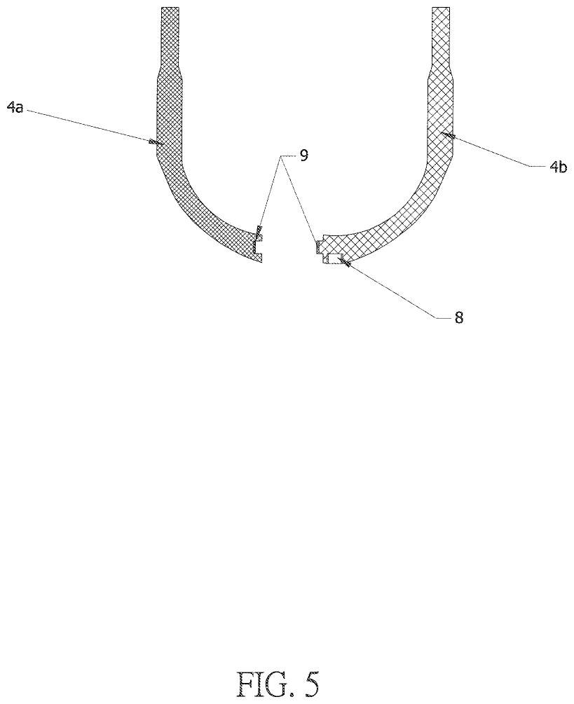

As shown in FIG. 2, FIG. 4 and FIG. 5, the medical silica gel stethoscope and the processing mold therefor provided in the Embodiment 2 of the present invention are basically identical with Embodiment 1, the only difference is the following structure: a corrugate pipe 10 is arranged in the straight pipe section, the inner pipe is a corrugate pipe 10, so that the silica gel of the U-section 3b is filled in the gaps and inner wall of corrugate pipe 10. A three-way pipe is embedded in the joint of the U-section and straight pipe section.

The present invention provides a method for manufacturing the medical silica gel stethoscope in the Embodiment 2, including the following steps:

1. Installation of sound conducting pipe mold: the straight pipe section core-pulling rod 2 is inserted into the straight pipe section 3a of sound conducting pipe mold 3, the outer end of straight pipe section core-pulling rod 2 is fixed to the outside of sound conducting pipe mold 3 by a lower fixing device. A U-core-pulling mold 4 is inserted into the U-section 3b of sound conducting pipe mold 3. Both ends of U-core-pulling mold 4 are fixed to an upper fixing device outside the mold. The end of the straight pipe section core-pulling rod 2 is inserted into the plug hole 5 of U-core-pulling mold 4. The sound conducting pipe mold 3 is clamped, so that a pouring space for silica gel pipe remains between the U-core-pulling mold 4 and the inner wall of sound conducting pipe mold 3. The lower side in the middle of the U-core-pulling mold 4 is provided with a threaded hole 8. The end of the straight pipe section core-pulling rod 2 has a threaded connection end with external thread matching the threaded hole 8. The threaded connection end of the straight pipe section core-pulling rod 2 enters the sound conducting pipe mold 3 and enters the threaded hole 8 to form threaded connection with the U-core-pulling mold 4. The U-core-pulling mold 4 includes a left core-pulling mold 4a and a right core-pulling mold 4b. The ends of left core-pulling mold 4a and right core-pulling mold 4b form inserting connection. The inserting connection ends of the left core-pulling mold 4a and right core-pulling mold 4b are provided with a set of magnetic attraction mechanisms 9 which can attract each other.

2. Molding of sound conducting pipe: the softened or liquid silica gel is injected to fill up the mold cavity with silica gel.

3. The sound conducting pipe mold 3 is dismounted after cooling, the straight pipe section core-pulling rod 2 and U-core-pulling mold 4 are pulled out, the surface of the molded sound conducting pipe 1 is polished.

4. The earhooks are inserted into both ends of U-section 1a, the stethoscope is connected to the end of straight pipe section.

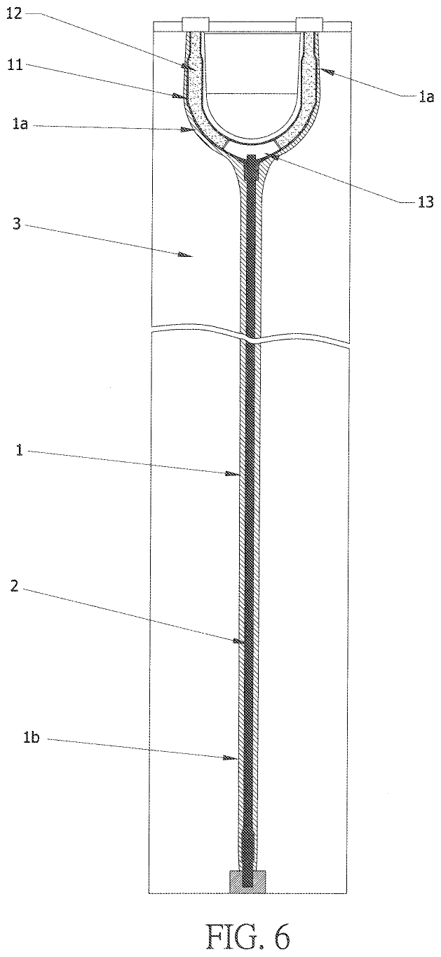

As shown in FIG. 6, the medical silica gel stethoscope provided in the Embodiment 3 of the present invention is basically identical with Embodiment 2, the only difference is that an inner pipe 11 is embedded in the U-section in advance, the inner pipe 11 is filled with a filler 12 to support the pattern of inner pipe 11. The filler 12 is withdrawn from the inner pipe 11 by dissolution, pumping or ejection after the U-section is molded by pouring.

To process the medical silica gel stethoscope of Embodiment 3, a three-way pipe 13 is embedded in the joint of the straight pipe section 1b and U-section 1a in advance, two inner pipes 11 are arranged in the U-section 1a, both ends of inner pipe are connected to the three-way pipe 13 and earhook respectively. During processing, the first step is to directly arrange the left and right inner pipes 11 and three-way pipe 13 in the sound conducting pipe mold, the inner pipe is filled with filler 12 to maintain the shape of inner pipe 11, and then the silica gel is filled between inner tube 11 and three-way pipe 13 and mold, cooled and molded, the filler 12 is removed from the inner pipe after the sound conducting pipe mold is removed. The filler 12 is a liquid or a gas, or a water soluble material, e.g. the mixture of salt and mud. The filler 12 can be discharged after watering. It can be sand, which can be poured out directly in late stage.

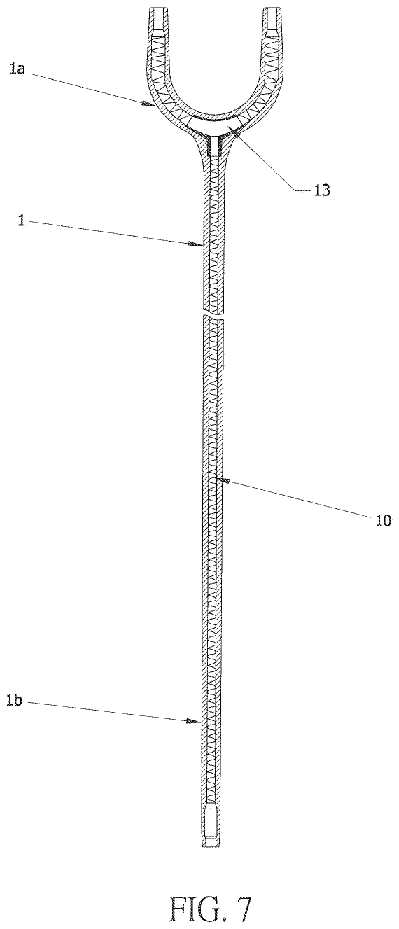

As shown in FIG. 7, the Embodiment 4 of the present invention is basically identical with Embodiment 2, the only difference is that both ends of the inner pipe are connected to the three-way pipe 13 and earhook respectively, the inner pipe is a corrugate pipe 10, the silica gel of U-section is filled in the gaps and inner wall of corrugate pipe 10.

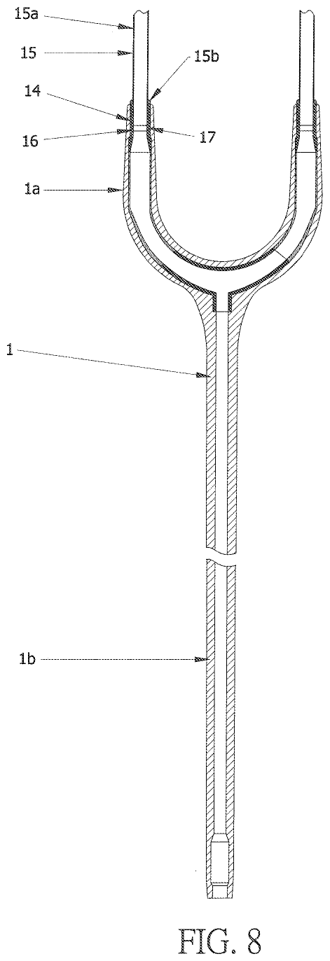

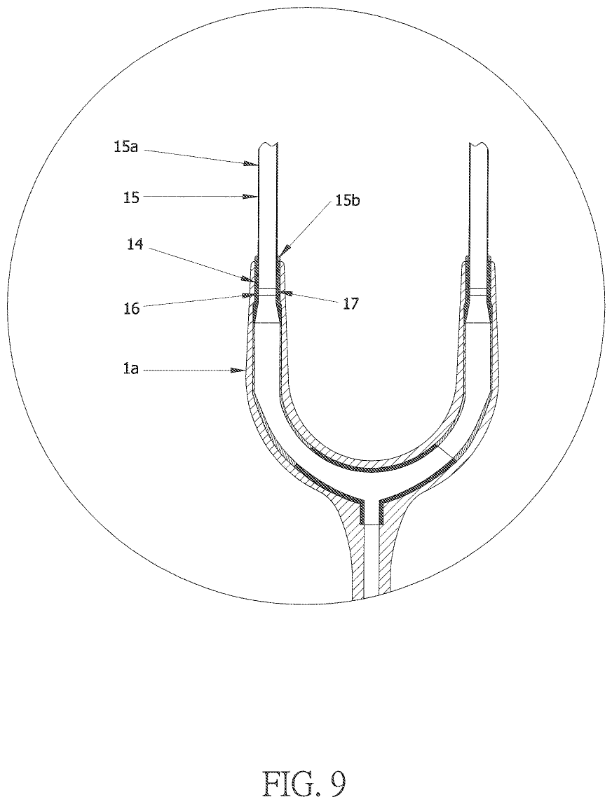

As shown in FIG. 8 and FIG. 9, the Embodiment 5 of the present invention is basically identical with Embodiment 1, the only difference is that the Embodiment 5 has the following structure: the earhook includes an earhook coupling end 14 and an earhook body 15. The earhook coupling end 14 is embedded in the U-section 1a in advance. The earhook body 15 is screwed with the earhook coupling end 14. An annular step 16 is arranged in the inner bore of earhook coupling end 14. A rubber sealing ring 17 is arranged on the annular step 16, the rubber sealing ring 17 is clamped between earhook body 15 and earhook coupling end 14. A nut 15b is screwed on the earhook pipe 15a of the earhook body 15 for adjusting the earhook locking position and direction. The nut 15b is located in the upside of the earhook coupling end 14, its height on the earhook pipe 15a is adjusted by turning the nut 15b, the length of earhook pipe 15a screwed in the earhook coupling end 14 can be adjusted, so as to adjust the locking position and direction, and the length of earhook pipe 15a can be adjusted.

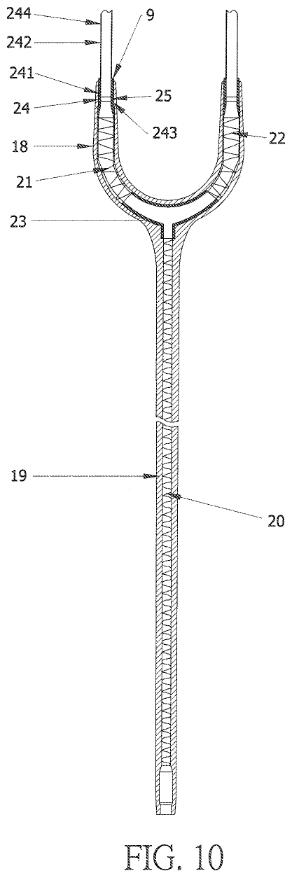

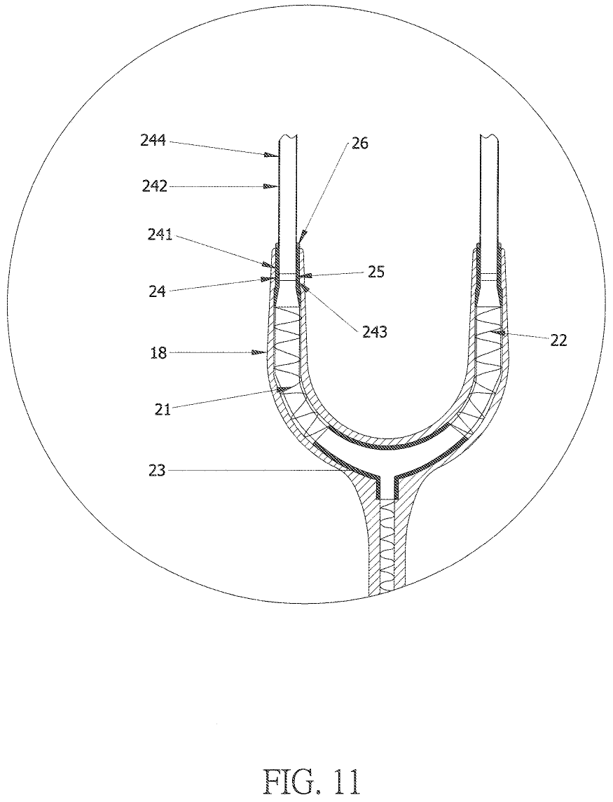

As shown in FIG. 10 and FIG. 11, the medical silica gel stethoscope provided in the Embodiment 6 of the present invention is basically identical with Embodiment 1, the only difference is that it includes a sound conducting pipe, an earhook 24 and a stethoscope. The sound conducting pipe includes a U-pipe section 18 and a straight pipe sound conducting section 19. The U-pipe section 18 and straight pipe sound conducting section 19 are molded integrally. A straight pipe corrugate pipe 20 is embedded in the inner wall of the straight pipe sound conducting section 19 in advance, so that the silica gel of the straight pipe sound conducting section 19 is filled in the gaps and inner wall of straight pipe corrugate pipe 20. A left corrugate pipe 21 and a right corrugate pipe 22 are embedded in right and left sides of the U-pipe section 18 respectively, so that the silica gel of U-pipe section 18 is filled in the gaps and inner walls of left corrugate pipe 21 and right corrugate pipe 22. The high-quality medical silica gel stethoscope can increase the hardness of inner wall of sound conducting pipe without losing its elasticity and flexibility, so as to enhance the sound conduction effect. The corrugate pipe can enhance the morphological stability of sound conducting pipe, favorable for maintaining better or restoring good pattern.

As shown in FIG. 11, the Embodiment 7 of the present invention is basically identical with Embodiment 6, the only difference is that the Embodiment 2 has the following structure: a three-way pipe 23 is embedded in the joint of the U-pipe section 18 and straight pipe sound conducting section 19 in advance, the inner end of the left corrugate pipe 21 contacts the left end of the three-way pipe 23, and the outer end contacts the inner end of the earhook 24. The inner end of the right corrugate pipe 22 contacts the right end of three-way pipe 23, and the outer end contacts the inner end of the earhook 24. In this embodiment, the three-way pipe 23 is favorable for the inner wall of sound conducting pipe to maintain smooth abutment after the embedded part is arranged.

As shown in FIG. 2, the earhook 24 includes an earhook coupling end 241 and an earhook body 242. The earhook coupling end 241 is embedded in the U-pipe section 18 in advance. The earhook body 242 is screwed with the earhook coupling end 241. An annular step 243 is arranged in the inner bore of earhook coupling end 241. A rubber sealing ring 25 is arranged on the annular step 243, the rubber sealing ring 25 is clamped between earhook body 242 and earhook coupling end 241. A nut 26 is screwed on the earhook pipe 244 of the earhook body 242 for adjusting the locking position and direction of earhook 24. The nut 26 is located in the upside of the earhook coupling end 241, its height on the earhook pipe 244 is adjusted by turning the nut 26, the length of earhook pipe 244 screwed in the earhook coupling end 241 can be adjusted, so as to adjust the locking position and direction, and the length of earhook pipe 244 can be adjusted.

We claim:

1. A medical silica gel stethoscope, including a sound conducting pipe, wherein the sound conducting pipe includes a U-section and a straight pipe section, the U-section and straight pipe section are molded integrally.

2. The medical silica gel stethoscope defined in claim 1, wherein a corrugate pipe is arranged in the straight pipe section.

3. The medical silica gel stethoscope defined in claim 1, wherein a three-way pipe is embedded in a joint of the U-section and straight pipe section.

4. The medical silica gel stethoscope defined in claim 3, wherein an inner pipe is embedded in the U-section, the inner pipe is filled with a filler to support the pattern of the inner pipe, the filler is withdrawn from the inner pipe by dissolution, pumping or ejection after the U-section is molded by pouring, both ends of the inner pipe are connected to the three-way pipe and earhook respectively, the inner tube is a corrugate pipe, so that the silica gel of the U-section is filled in the gaps and inner wall of the corrugate pipe.

5. The medical silica gel stethoscope defined in claim 1, a straight pipe corrugate pipe is embedded in the inner wall of the straight pipe section, so that the silica gel of straight pipe section is filled in the gaps and inner wall of straight pipe corrugate pipe, a left corrugate pipe and a right corrugate pipe are embedded in the right and left sides of the U-section respectively, so that the silica gel of U-section is filled in the gaps and inner walls of left corrugate pipe and right corrugate pipe, a three-way pipe is embedded in the joint of the U-section and straight pipe section, the inner end of the left corrugate pipe contacts the left end of three-way pipe, and the outer end contacts the inner end of earhook, the inner end of the right corrugate pipe contacts the right end right end of three-way pipe, and the outer end contacts the inner end of earhook, the earhook includes an earhook coupling end and an earhook body, the earhook coupling end is embedded in the U-section, the earhook body is screwed with the earhook coupling end, an annular step is arranged in the inner bore of earhook coupling end, a rubber sealing ring is arranged on the annular step, the rubber sealing ring is clamped between the earhook body and earhook coupling end, the earhook pipe of the earhook body is provided with a nut to adjust the earhook locking position and direction. The nut is located in the upside of earhook coupling end.

6. A manufacturing method for the medical silica gel stethoscope defined in claim 1 includes the following steps:
installing the sound conducting pipe mold, wherein the straight pipe section core-pulling rod is inserted into the straight pipe section of sound conducting pipe mold, the outer end of straight pipe section core-pulling rod is fixed to the outside of sound conducting pipe mold by a lower fixing device, a U-core-pulling mold is inserted into the U-section of sound conducting pipe mold, both ends of U-core-pulling mold are fixed to the upper fixing device outside the mold, the end of the straight pipe section core-pulling rod is inserted into the plug hole of U-core-pulling mold, the sound conducting pipe mold is clamped, a pouring space for silica gel pipe remains between the U-core-pulling mold and the inner wall of sound conducting pipe mold;
molding the sound conducting pipe: the softened or liquid silica gel is injected to fill up the mold cavity with silica gel;
removing the sound conducting pipe mold after cooling, the straight pipe section core-pulling rod and U-core-pulling mold are pulled out, the surface of the molded sound conducting pipe is polished;
inserting the earhooks into both ends of U-section, the stethoscope is connected to the end of straight pipe section.

7. The manufacturing method for the medical silica gel stethoscope defined in claim 6, wherein a threaded hole is arranged in the downside in the middle of U-core-pulling mold in Step 1, the end of the straight pipe section core-pulling rod has a threaded connection end with external thread matching the threaded hole, the threaded connection end of the straight pipe section core-pulling rod enters the sound conducting pipe mold and enters the threaded hole to form threaded connection with the U-core-pulling mold.

8. The manufacturing method for the medical silica gel stethoscope defined in claim 7, wherein the U-core-pulling mold includes a left core-pulling mold and a right core-pulling mold, the ends of left core-pulling mold and right core-pulling mold form inserting connection, the inserting connection ends of the left core-pulling mold and right core-pulling mold are provided with a set of magnetic attraction mechanisms which can attract each other.

9. The manufacturing method for the medical silica gel stethoscope defined in claim 8, wherein a three-way pipe is embedded in the joint of the straight pipe section and U-section, two inner pipes are arranged in the U-section, both ends of inner pipe are connected to the three-way pipe and earhook respectively, during processing, the first step is to directly place the left and right inner pipes and three-way pipe in the sound conducting pipe mold, the inner pipe is filled with a filler to maintain the shape of inner pipe, and the silica gel is filled in between the inner pipe and three-way pipe and mold, cooled and molded, the filler is removed from the inner pipe after the sound conducting pipe mold is removed.

10. A processing mold for the medical silica gel stethoscope defined in claim 1 includes a sound conducting pipe mold, wherein the sound conducting pipe mold includes an upper mold, a lower mold, a straight pipe section core-pulling rod and a U-core-pulling mold, the end of straight pipe section of the sound conducting pipe mold is provided with a lower plug, both ends of the U-section are provided with an upper plug respectively, the lower end of the straight pipe section core-pulling rod is provided with a fixing device, both ends of the U-core-pulling mold are provided with a fixing device respectively, the lower fixing device is a lower plug, the straight pipe section core-pulling rod is arranged on the lower plug, the upper end of the straight pipe section core-pulling rod is provided with a threaded connection end, the upper fixing device is an upper plug, both ends of the U-core-pulling mold are arranged on the upper plug respectively, a threaded hole is arranged in the downside in the middle of the U-core-pulling mold, the end of the straight pipe section core-pulling rod has a threaded connection end with external thread matching the threaded hole, the threaded connection end of the straight pipe section core-pulling rod enters the sound conducting pipe mold and enters the threaded hole to form threaded connection with the U-core-pulling mold, the lower end of the straight pipe section core-pulling rod is fixed to the lower plug, both ends of the U-core-pulling mold are fixed to the lower plug, the U-core-pulling mold includes a left core-pulling mold and a right core-pulling mold, the ends of left core-pulling mold and right core-pulling mold form inserting connection, the inserting connection ends of the left core-pulling mold and right core-pulling mold are provided with a set of magnetic attraction mechanisms which can attract each other.
